(19) United States
(12) Patent Application Publication
(10) Pub. No.: US 2011/0195042 A1
(43) Pub. Date: Aug. 11, 2011

(54) Compositions, Methods and Kits Useful for Treating a Respiratory Symptom

(76) Inventors: Thomas Edward Huetter, West Chester, OH (US); Tracy L. Grosick, West Chester, OH (US)

(21) Appl. No.: 13/021,903

(22) Filed: Feb. 7, 2011

Related U.S. Application Data
(60) Provisional application No. 61/302,981, filed on Feb. 10, 2010.

(51) Int. Cl. A61K 31/765; A61K 31/715; A61K 31/79; A61K 31/78; A61K 31/277; A61K 31/165; A61K 31/485; A61P 11/00; A61P 11/14

(52) U.S. Cl. 424/78.32; 424/78.31; 424/78.37; 514/54; 424/195.18; 514/521; 514/617; 514/289

(57) ABSTRACT

The present disclosure describes methods for enhancing an overall cooling sensation of an oral mucosa comprising administering a liquid composition comprising a thickening agent and/or a mucoadhesive polymer, a non-menthol coolant; and contacting the oral mucosa with the liquid composition. The disclosure also describes the liquid compositions.

### CROSS REFERENCE TO RELATED APPLICATIONS

This reference claims the benefit of U.S. Provisional Application No. 61/302,981, filed Feb. 10, 2010.

### FIELD OF THE INVENTION

The present invention relates to a method treating a respiratory symptom comprising administering a liquid composition. The invention further relates to the liquid compositions as described herein.

### BACKGROUND OF THE INVENTION

Currently, consumers seeking relief from respiratory symptoms (e.g., symptomatic relief, whether temporary or permanent) including relief from sore throat, post nasal drip, or general cold symptoms experience a lag time between when consumers take orally ingested respiratory products and when they start to feel relief from the symptoms of a cold. This is because most respiratory products contain actives that must become bio-available in the bloodstream before they take effect. Therefore, a consumer must wait for a product to become absorbed in order to have any alleviation of symptoms.

As an example, cough syrups often contain actives that must be absorbed by the blood wherein often about 30 minutes elapses prior to cough relief; therefore there is no instant or extended sensory/olfactory experience that is targeted to provide the consumer the perception of “feeling better” during this therapeutic lag time.

The present invention provides a liquid composition that provides an immediate and extended cooling sensation delivered through increased coating of the oral mucosa such as the mouth and throat; the composition comprises a system that promotes increased contact time with the oral mucosa through the synergy of the polymers comprised in the composition and the type of coolants that are selected. Additionally, the liquid composition can be used to enhance and/or modulate the coolant that is incorporated into the composition which targets the oral mucosa and provide cooling sensation that lasts greater than 10 minutes and results in a consumer reaction of “feeling better” faster.

### SUMMARY OF THE INVENTION

One embodiment is directed to a method of enhancing an overall cooling sensation of an oral mucosa comprising the steps of; administering a liquid composition comprising: 1) a thickening agent; 2) optionally a mucoadhesive polymer; and 3) a non-menthol coolant; and contacting the oral mucosa with the liquid composition.

An additional embodiment further relates to a method of increasing the contact time between the liquid composition and an oral mucosa to increase an overall cooling sensation comprising the steps of administering a liquid composition comprising: 1) a thickening agent; 2) optionally a mucoadhesive polymer; and 3) a non-menthol coolant; and contacting the oral mucosa with the liquid composition.

One embodiment is directed to a method of enhancing an overall cooling sensation of an oral mucosa comprising the steps of; administering a liquid composition comprising: 1) a mucoadhesive polymer; 2) optionally a thickening agent; and 3) a non-menthol coolant; and contacting the oral mucosa with the liquid composition.

An additional embodiment is further directed to a method of enhancing and/or modulating activity of one or more non-menthol coolants incorporated into a liquid composition comprising the steps of; formulating a liquid composition comprising: 1) a thickening agent; 2) optionally a mucoadhesive polymer; and 3) a non-menthol coolant; administering the liquid composition; contacting the oral mucosa with the liquid composition; whereby the liquid composition provides upon contact with the oral mucosa an extended cooling sensation which lasts greater than 10 minutes.

An additional embodiment is further directed to a method of providing a soothing effect to a user experiencing the symptoms of a cold comprising the steps of: administering a liquid composition comprising 1) a thickening agent; 2) optionally a mucoadhesive polymer; and 3) a non-menthol coolant; contacting the liquid composition with the users oral mucosa; and delivering an overall cooling sensation to the oral mucosa of the user.

An additional embodiment is further directed to a liquid composition that delivers an overall cooling sensation to the oral mucosa of a user comprising, a thickening agent; a mucoadhesive polymer; and N-(4-cyanomethylphenyl)-ρ-menthanecarboxamide; wherein the composition provides an extended cooling sensation that lasts greater than 10 minutes.

An additional embodiment is further directed to a method of increasing the contact time between a liquid composition and an oral mucosa to increase an overall cooling sensation comprising the steps of administering a liquid composition comprising: 1) a mucoadhesive polymer; 2) optionally a thickening agent; and 3) a non-menthol coolant; and contacting the oral mucosa with the liquid composition.

An additional embodiment is further directed to a method of enhancing and/or modulating activity of one or more non-menthol coolants incorporated into a liquid compositions comprising the steps of: formulating a liquid composition comprising: 1) a mucoadhesive polymer; 2) optionally a thickening agent; and 3) a non-menthol coolant; administering the liquid composition; contacting the oral mucosa with the liquid composition; whereby the liquid composition provides upon contact with the oral mucosa an extended cooling sensation that lasts greater than 10 minutes.

An additional embodiment is further directed to a method of providing a soothing effect to a user experiencing the symptoms of a cold comprising the steps of; administering a liquid composition comprising: 1) a mucoadhesive polymer; 2) optionally a thickening agent; 3) a non-menthol coolant; and contacting the liquid composition with the user's oral mucosa; and delivering an overall cooling sensation to the oral mucosa of the user.

### BRIEF DESCRIPTION OF THE DRAWINGS

FIG. 1 is a graph of Perceived Product thickness;

FIG. 2 is a graph of Perceived Oral cooling intensity as a function of time;

FIG. 3 is a graph of Perceived Throat cooling intensity as a function of time;

FIG. 4 is a graph of Perceived Overall Cooling sensation intensity as a function of time; and

FIG. 5 is a graph of Perceived Soothing intensity as a function of time.

### DETAILED DESCRIPTION OF THE INVENTION

The present invention is directed to a method of treating a respiratory symptom comprising the steps of administering a liquid composition comprising: 1) a thickening agent; 2) optionally a mucoadhesive polymer; and 3) a non-menthol coolant; and contacting the oral mucosa with the liquid composition.

These and other limitations of the compositions and methods of the present invention, as well as many of the optional ingredients suitable for use herein, are described in detail hereinafter.

The term “overall cooling sensation” as used herein means an amount of heating, cooling, numbing, and tingling sensations penetrating into the tissues of the oral mucosa, throat, and chest cavities.

The term “liquid composition” as used herein refers to a composition in a form that is deliverable to a mammal in need thereof via the oral cavity, mouth, throat, nasal passage or combinations thereof. A liquid composition can be in a form that is directly deliverable to the oral mucosa.

The term “oral mucosa” as used herein refers to mouth and throat. These compositions can be optionally delivered by a delivery device selected from droppers, pumps, sprayers, liquid droppers, spoons, cups, squeezable sachets, power shots, and other containers, devices, packaging or equipment, and combinations thereof.

All weights, measurements and concentrations herein are measured at 25° C. on the composition in its entirety, unless otherwise specified.

All percentages, parts and ratios as used herein are by weight of the total composition, unless otherwise specified. All such weights as they pertain to listed ingredients are based on the active level and, therefore do not include solvents or by-products that may be included in commercially available materials, unless otherwise specified.

The composition, preparations and methods of the present invention can comprise, consist of, or consist essentially of, the essential elements and limitations of the invention described herein, as well as any additional or optional ingredients, components, or limitations described herein or otherwise useful in compositions intended for use or consumption by mammals preferably consumption or use by humans.

### Methods of the Present Invention

The present invention herein is directed to methods of treating a respiratory symptom in a mammal (such as, for example, a human) in need of such treatment including any of the following: enhancing cooling sensation of an oral mucosa; increasing the contact time between a liquid composition and an oral mucosa; modulating activity of one or more coolants; and providing a soothing effect to a mammal experiencing a cold symptom, for example, sore, dry or otherwise discomfort in the throat or other areas of the oral mucosa.

In one embodiment, the methods of the present invention provide for a means of enhancing the overall cooling sensation of an oral mucosa. The methods include the administration of the liquid composition to provide a coating action of the oral mucosa that increases the contact time of the composition with the oral mucosa and allows for an immediate and an extended cooling sensation. This increase in cooling sensation is due to the synergy present between the various ingredients comprised in the liquid composition such as, for example, thickening agents, mucoadhesive polymers, or the non-menthol coolants chosen.

As used herein, “enhancing” and/or “providing relief” with respect to the cooling sensation means that administration of the referenced respiratory composition provides an immediate and or extended sensation of cooling which provides the perception of alleviation, amelioration, inhibition, or mitigation of one or more symptoms of respiratory symptoms to the mammal.

In a further embodiment herein, the present invention is directed to methods of enhancing an overall cooling sensation of an oral mucosa comprising the steps of: administering a liquid composition comprising: a thickening agent and/or a mucoadhesive polymer; and a non-menthol coolant; and contacting the oral mucosa with the liquid composition.

In a further embodiment, the invention is directed to a method of increasing the contact time between a liquid composition and an oral mucosa to increase an overall cooling sensation comprising the steps of administering a liquid composition comprising a thickening agent and/or a mucoadhesive polymer; and a non-menthol coolant; and contacting the oral mucosa with the liquid composition.

In a further embodiment, the present invention is directed to a method of modulating the activity of one or more non-menthol coolants incorporated into a liquid composition comprising the steps of: formulating a liquid composition comprising a thickening agent and/or a mucoadhesive polymer; and a non-menthol coolant; administering the respiratory composition; and contacting the oral mucosa with the liquid composition; whereby the liquid composition provides upon contact with the oral mucosa an extended cooling sensation which lasts greater than 10 minutes. The term “modulating” is used herein to mean modifying the effect of the one or more non-menthol coolants contained in the liquid composition that aids in tempering harsh, burning, biting, or bitter sensations from the coolant(s), including but not limited to enhancing its activity, so as to provide an extended cooling sensation that lasts greater than 10 minutes, alternatively greater than 20 minutes after administration, alternatively greater than 30 minutes after administration, alternatively greater than 45 minutes after administration, alternatively greater than 60 minutes after administration.

In a further embodiment, the present invention is directed to a method of providing a soothing effect to a mammal experiencing the symptoms of a cold comprising the steps of: administering a liquid composition comprising: 1) a thickening agent; 2) a mucoadhesive polymer; 3) a non-menthol coolant; contacting the liquid composition with the mammal's oral mucosa; and delivering an overall cooling sensation to the oral mucosa of the user.

As used herein, the term “orally administering” and/or “administering” with respect to the human/mammal means that the human/mammal ingests or is directed to ingest (whether by swallowing, spraying or any other means) one or more of the present liquid compositions. The human/mammal may be directed to deliver the liquid composition to the site that the human/mammal intends to treat, for example, the oral mucosa. The human/mammal may be directed to ingest the composition, and such direction and or delivery may be that which instructs and/or informs the human that use of the composition may and/or will provide the perception of relief from the respiratory symptom (e.g., symptomatic relief, whether temporary or permanent) for example, relief from sore throat. The relief can be instant or extended. For example, such direction may be oral direction (e.g., through oral instruction from, for example, a physician, pharmacist, or other health professional), radio or television media (e.g., advertisement), or written direction (e.g., through written direction from, for example, a physician, pharmacist, or other health professional (e.g., scripts), sales professional organization (e.g., through, for example, marketing brochures, pamphlets, or other instructive paraphernalia), written media (e.g., internet, electronic mail, or other computer-related media), and/or packaging associated with the composition (e.g., a label present on a delivery device holding the composition). As used herein, “written” means through words, pictures, symbols, and/or other visible or tactile descriptors. Such information need not utilize the actual words used herein, for example, “respiratory”, “symptom”, or “mammal”, but rather use of words, pictures, symbols, tactile means, and the like conveying the same or similar meaning are contemplated within the scope of this invention.

Administration may be on an as-needed or as-desired basis, for example, once-monthly, once-weekly, or daily, including multiple times daily, for example, at least once daily, from one to about forty times daily, from one to about thirty times daily, from one to about twenty times daily, from one to about fifteen times daily, from one to about ten times daily, from about two to about four times daily, or about three times daily.

The amount of liquid composition administered may be dependent on a variety of factors, including the general quality of health of the mammal, age, gender, weight, or severity of symptoms.

### Liquid Compositions of the Present Invention

In one embodiment a liquid composition of the present invention comprises: 1) a thickening agent; 2) optionally a mucoadhesive polymer; and 3) a non-menthol coolant.

In one embodiment, the composition is a non-Newtonian liquid that exhibits zero shear viscosity from about 100 centiPoise (cP) to about 1,000,000 cP, from about 100 cP to about 500,000 cP, from about 100 cP to about 100,000 cP, from about 100 cP to about 50,000 cP, from about 200 cP to about 20,000 cP, from about 600 cP to about 20,000 cP, from about 1,000 to about 10,000 cP at a temperature of 37±1° C., as measured according to the Shear Viscosity Method described hereafter. The composition may exhibit desirable dosing characteristics, such as pourability, dose accuracy, and low cup retention, thinning upon swallowing and thickening when shear is removed, providing increased contact time for coating and extended and immediate cooling. It is believed that there is a synergy based on the product thickness due to entanglement of the polymer chains and the non-menthol coolant to provide the extended cooling sensation.

In one embodiment, the liquid composition has a pH of from about 1 to about 7, from about 2 to about 6.5, and from about 4 to about 6.

### Thickening Agent

The liquid composition of the present invention can comprise a thickening agent. When present, the composition comprises from about 0.01% to about 10% thickening agent, alternatively from about 0.02% to about 5%, alternatively from about 0.03% to about 4%, alternatively from about 0.04% to about 3%, alternatively from about 0.05% to about 1% thickening agent, by weight of the composition.

Nonlimiting examples of thickening agents include xanthan gum, cellulosic polymers such as carboxymethycellulose (CMC), hydroxethylcellulose, hydroxymethylcellulose, and hydroxypropylmethylcellulose, carrageenan, polyacrylic acid, cross-linked polyacrylic acid such as Carbopol®, polycarbophil, alginate, clay, and combinations thereof. In one embodiment, the thickening agent is xanthan gum.

### Mucoadhesive Polymer

The liquid composition of the present invention can comprise a mucoadhesive polymer. When present, the composition comprises from about 0.01% to about 10% mucoadhesive polymer, alternatively from about 0.02% to about 5%, alternatively from about 0.03% to about 4%, alternatively from about 0.04% to about 3%, alternatively from about 0.05% to about 3%, by weight of the composition.

Nonlimiting examples of mucoadhesive polymer include polyvinylpyrrolidone (Povidone), methyl vinyl ether copolymer of maleic anhydride (Gantrez®), guar gum, gum tragacanth, polydextrose, cationic polymers, poly(ethylene oxide), poly(ethylene glycol), poly(vinyl alcohol), poly(acrylic acid), cross-linked polyacrylic acid such as Carbopol®, polycarbophil, poly(hydroxyl ethyl methacrylate), chitosan, cellulosic polymers such as carboxymethycellulose (CMC), hydroxethylcellulose, hydroxymethylcellulose, hydroxypropylmethylcellulose, and combinations thereof. In one embodiment, the mucoadhesive polymer is polyvinylpyrrolidone.

### Non-Menthol Coolant

The liquid composition of the present invention can comprise a non-menthol coolant. The non-menthol coolant is not menthol, however, the non-menthol coolants can comprise a structural modification of menthol. It is desirable that the coolants possess a distinctive odor or flavor while providing a cool sensation of extended duration, in order that the effect can still be perceived for a considerable time after contact with the oral mucosa, for example, for at least 10 minutes, alternatively greater than 20 minutes after administration, alternatively greater than 30 minutes after administration, alternatively greater than 45 minutes after administration, alternatively greater than 60 minutes after administration.

When present, the composition comprises from about 0.00001% to about 2% non-menthol coolant, alternatively from about 0.00005% to about 1%, alternatively from about 0.001% to about 1%, alternatively from about 0.001% to about 0.5%, alternatively from about 0.001% to about 0.03% non-menthol coolant, by weight of the composition.

Non-limiting examples of non-menthol coolants include menthone glycerol acetal (for example, sold as Frescolat® MGA by Haarmann & Reimer), N-(4-cyanomethylphenyl)-ρ-menthanecarboxamide or N-(4-cyanomethylphenyl)-5-methyl-2-(1-methylethyl)cyclohexanecarboxamide (for example, commercially available from Givaudan), N-(2-(pyridin-2-yl)ethyl-3-ρ-menthanecarboxamide (for example, commercially available from Givaudan), N-(4-sulfamoylphenyl)-ρ-menthanecarboxamide, N-(4-cyanophenyl)-ρ-menthanecarboxamide, N-(4-acetylphenyl)-ρ-menthanecarboxamide, N-(4-hydroxymethylphenyl)-ρ-menthanecarboxamide, N-(3-hydroxy-4-methoxyphenyl)-ρ-menthanecarboxamide, 2-Isopropyl-N,2,3-trimethylbutyramide (for example, known as WS-23); N-Ethyl-ρ-menthane-3-carboxamide (for example, known as WS-3); Ethyl 3-(ρ-menthane-3-carboxamido)acetate (for example, known as WS-5), menthyl lactate (for example, commercially available as Frescolat® ML by Haarmann & Reimer), Menthoxypropane-1,2-diol (for example, commercially available as Coolant Agent 10 by Takasago International), ρ-Menthane-3,8-diol (for example, commercially available as PMD38)-Takasago International, Isopulegol (for example, commercially available under the name “Coolact P®” by Takasago International), (1R,2S,5R)-2-isopropyl-5-methyl-N-(2-(pyridyn-2-yl)ethylcyclohexane carboxamide, (1-glyceryl-p-mentane-3-carboxylate), (ethyleneglycol-p-methane-3-carboxylate), (N-t-butyl-p-menthane-3-carboxamide), (N-(4-,ethoxyphenyl)-p-menthane-3-carboxamide), 3-(1-menthoxy)propane-1,2-diol, 3-(1-Menthoxy)-2-methylpropane-1,2-diol, menthyl pyrrolidone carboxylate) (for example, commercially available as Questice®), (1R,3R,4S)-3-menthyl-3,6-dioxaheptanoate (for example, commercially available from Firmenich), (1R,2S,5R)-3-menthyl methoxyacetate (for example, commercially available from Firmenich), (1R,2S,5R)-3-menthyl 3,6,9-trioxadecanoate (for example, commercially available from Firmenich), (1R,2S,5R)-menthyl 11-hydroxy-3,6,9-trioxaundecanoate (for example, commercially available from Firmenich), (1R,2S,5R)-3-menthyl (2-hydroxyethoxy)acetate (for example, commercially available from Firmenich), Cubebol (for example, commercially available from Firmenich), 1-[2-hydroxyphenyl]-4-[2-nitrophenyl-]-1,2,3,6-tetrahydropyrimidine-2-one), 4-methyl-3-(1-pyrrolidinyl)-2[5H]-furanone (for example, known as Icilin or AG-3-5), menthyl lactate, menthone glycerin acetal, L-Monomenthyl succinate, L-monomenthyl glutarate, 3-1-menthoxypropane-1,2-diol (for example, known as Coolact 10), 2-1-menthoxyethanol (for example, known as Cooltact 5), and mixtures thereof. Additional non-menthol coolants are described in U.S. Pat. No. 7,414,152, US20100086498 A1 and WO2010/128026 A2. In one embodiment, the non-menthol coolant is N-(4-cyanomethylphenyl)-ρ-menthanecarboxamide including all 8 stereoisomers arising from the 3 chiral centers. In particular, the [1R, 2S, 5R]-N-(4-cyanomethylphenyl)-ρ-menthanecarboxamide can be readily synthesized from natural 1-menthol.

### Flavoring Ingredient

The non-menthol coolant may be supplied in the composition as single or purified chemicals or by addition of a flavoring ingredient such as natural oils or extracts. In one embodiment such natural oils or extracts may have been refined to remove components that are relatively unstable and may degrade and alter the desired sensate profile, resulting in a less acceptable product from an organoleptic standpoint. When present, flavoring ingredients are generally used in the compositions at levels of from about 0.001% to about 8%, by weight of the composition. Nonlimiting examples include EverCool™ 180 available from Givaudan of Cincinnati, Ohio which is available, for example, as a 5% solution of N-(4-cyanomethylphenyl)-ρ-menthanecarboxamide in a flavoring ingredient cool white grape, or as a 7.5% solution of N-(4-cyanomethylphenyl)-ρ-menthanecarboxamide in a flavor ingredient such as spearmint or peppermint. Additional examples of flavoring ingredients include but are not limited to peppermint oil, corn mint oil, spearmint oil, oil of wintergreen, clove bud oil, cassia, sage, parsley oil, marjoram, lemon, lime, orange, cherry, cis-jasmone, 2,5-dimethyl-4-hydroxy-3(2H)-furanone, 5-ethyl-3-hydroxy-4-methyl-2(5H)-furanone, vanillin, ethyl vanillin, anisaldehyde, 3,4-methylenedioxybenzaldehyde, 3,4-dimethoxybenzaldehyde, 4-hydroxybenzaldehyde, 2-methoxybenzaldehyde, benzaldehyde; cinnamaldehyde, hexyl cinnamaldehyde, alpha-methyl cinnamaldehyde, ortho-methoxy cinnamaldehyde, alpha-amyl cinnamaldehyde, propenyl guaethol, heliotropine, 4-cis-heptenal, diacetyl, methyl-ρ-tert-butyl phenyl acetate, menthol, methyl salicylate, ethyl salicylate, 1-menthyl acetate, oxanone, alpha-irisone, methyl cinnamate, ethyl cinnamate, butyl cinnamate, ethyl butyrate, ethyl acetate, methyl anthranilate, iso-amyl acetate, iso-amyl butyrate, allyl caproate, eugenol, eucalyptol, thymol, cinnamic alcohol, octanol, octanal, decanol, decanal, phenylethyl alcohol, benzyl alcohol, alpha-terpineol, linalool, limonene, citral, maltol, ethyl maltol, anethole, dihydroanethole, carvone, menthone, β-damascenone, ionone, gamma decalactone, gamma nonalactone, gamma undecalactone and mixtures thereof. Generally suitable flavoring ingredients are those containing structural features and functional groups that are less prone to redox reactions. These include derivatives of flavor chemicals that are saturated or contain stable aromatic rings or ester groups.

### Additional Component

The liquid composition can comprise one or more additional components. When present, the composition comprises from about 0.00001% to about 30%, alternatively from about 0.0001% to about 25%, alternatively from about 0.001% to about 20%, alternatively from about 0.01% to about 15%, alternatively from about 0.1% to about 10%, alternatively from about 1% to about 10% of additional components, by weight of the composition of additional components.

Nonlimiting examples of additional components include cooling agents such as menthol, warming agents, flavoring agents, salivating agents, tea extract, Vitamin A, Vitamin C, Vitamin B, Vitamin D, carotenoid, rosemary, rosemary extract, caffeic acid, coffee extract, tumeric extract, curcumin, blueberry extract, grapeseed extract, rosemaric acid, antioxidant, amino acid, enzyme, prebiotic, probiotic, andrographis extract, 1-tryptophan, Allium sativum, herbal remedies, vitamins, supplements, antioxidants, natural ingredients, minerals, energy boosting ingredients, sleep aids, immune system boosting agents, colorant, preservative, fragrance, flavorant, fruit extract, a salivating stimulator, and combinations thereof.

Nonlimiting examples of cooling agents include, menthol, (1-glyceryl-ρ-mentane-3-carboxylate) (for example, known as WS-30), (ethyleneglycol-p-methane-3-carboxylate) (for example, known as WS-30), (N-t-butyl-p-menthane-3-carboxamide) (for example, known as WS-14), (N-(4-,ethoxyphenyl)-p-menthane-3-carboxamide) (for example, known as WS-12), 3-(1-menthoxy)propane-1,2-diol, 3-(1-Menthoxy)-2-methylpropane-1,2-diol, menthyl pyrrolidone carboxylate (for example, commercially available as Questice®), (1R,3R,4S)-3-menthyl-3,6-dioxaheptanoate (for example, commercially available from Firmenich), (1R,2S,5R)-3-menthyl methoxyacetate (for example, commercially available from Firmenich), (1R,2S,5R)-3-menthyl 3,6,9-trioxadecanoate (for example, commercially available from Firmenich), (1R,2S,5R)-menthyl 11-hydroxy-3,6,9-trioxaundecanoate (for example, commercially available from Firmenich), (1R,2S,5R)-3-menthyl (2-hydroxyethoxy)acetate (for example, commercially available from Firmenich), Cubebol (for example, commercially available from Firmenich), 1-[2-hydroxyphenyl]-4-[2-nitrophenyl-]-1,2,3,6-tetrahydropyrimidine-2-one (for example, known as Icilin or AG-3-5), 4-methyl-3-(1-pyrrolidinyl)-2[5H]-furanone, menthyl lactate, menthone glycerin acetal, Peppermint oil, L-Monomenthyl succinate, L-monomenthyl glutarate, 3-1-menthoxypropane-1,2-diol (for example, known as Coolact 10), and 2-1-menthoxyethanol (for example, known as Coolact 5).

Nonlimiting examples of warming agents include TK 1000, TK 1 mM, Heatenol (commercially available from Sensient Flavors, Optaheat (commercially available from Symrise Flavors), Cinnamon, Polyethylene glycol, Capsicum, Capsaicin, and Curry.

Nonlimiting examples of flavoring agents include natural flavoring agents, artificial flavoring agents, artificial extracts, natural extracts and combination thereof. Nonlimiting examples of flavoring agents include: Vanilla, honey lemon, lemon honey, cherry vanilla, peach, honey ginger, chamomile, cherry, cherry cream, mint, vanilla mint, dark berry, black berry, raspberry, peppermint, spearmint, honey peach, acai berry, cranberry, honey cranberry, tropical fruit, dragon fruit, wolf berry, red stem mint, pomegranate, black current, strawberry, lemon, lime, peach ginger, orange, orange cream, creamsicle, apricot, anethole, ginger, jack fruit, star fruit, blueberry, fruit punch, lemon grass, chamomile lemon grass, lavender, banana, strawberry banana, grape, blue raspberry, lemon lime, coffee, espresso, cappuccino, honey, wintergreen mint, bubble gum, tart honey lemon, sour lemon, green apple, boysenberry, rhubarb, strawberry rhubarb, persimmon, green tea, black tea, red tea, white tea, honey lime, cherry lime, apple, tangerine, grapefruit, kiwi, pear, vanillin, ethyl vanillin, maltol, ethyl-maltol, pumpkin, carrot cake, white chocolate raspberry, chocolate, white chocolate, milk chocolate, dark chocolate, chocolate marshmallow, apple pie, cinnamon, hazelnut, almond, cream, crème Brule, caramel, caramel nut, butter, butter toffee, caramel toffee, aloe Vera, whiskey, rum, cocoa, licorice, pineapple, guava, melon, watermelon, elder berry, mouth cooler, raspberries and cream, peach mango, tropical, cool berry, lemon ice, nectar, spicy nectar, tropical mango, apple butter, peanut butter, tangerine, tangerine lime, marshmallow, cotton candy, apple cider, orange chocolate, and mixtures thereof.

Nonlimiting examples of salivating agents include formula (I):

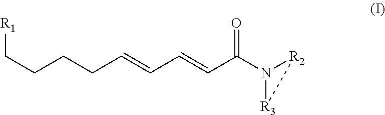

wherein R₁ represents C1-C2 n-alkyl; R₂ is 2-methyl-1-propyl and R₃ is hydrogen, or R₂ and R₃ taken together is a moiety having the formula —(CH₂)ₙ— wherein n is 4 or 5, or mixtures thereof.

In one embodiment, the salivating agent comprises a material wherein R₂ is 2-methyl-1-propyl and R₃ is hydrogen, more preferably wherein R₁ is C1 n-alkyl, R₂ is 2-methyl-1-propyl and R₃ is hydrogen. More preferably, the salivating agent comprises trans-pellitorin, a chemical having a structure according to formula (II):

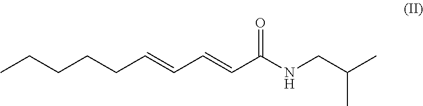

The preferred form of Vitamin C for use in the liquid composition is as ascorbic acid or the equivalent of a salt of ascorbic acid or the equivalent of a derivative of ascorbic acid. The vitamin C may either be in an immediate release form or a sustained release form.

Vitamin A and carotene can be obtained from either animal or vegetable sources. The vitamin A can be in the form of vitamin A, retinol, retinyl palmitate, retinyl acetate, retinyl proprionate, beta-carotene, alpha carotene, beta-cryptoxanthin, and mixtures thereof.

Nonlimiting examples of Vitamin D include Vitamin D3 (cholecalciferol), Vitamin D2 (ergocalciferol) and combinations thereof. Additional, nonlimiting examples also include metabolites of Vitamin D, including calcidiol, calcitriol, and combinations thereof. The Vitamin D, including cholecalciferol, ergocalciferol, calcidiol and calcitriol, may be derived from synthetic or natural sources. Vitamin D, including cholecalciferol and calcitriol, may be sourced from an extract of solanum glaucophyllum (malacoxylon), trisetum flavescens (goldhafer) or cestrum diurnum. Both the pure, Vitamin D and/or glycosides of the Vitamin D, may be used.

Tea extract is a polyphenol. Nonlimiting examples of extracts includes Camellia sinensis. Nonlimiting sources of tea extract for use in the present invention are black tea, white tea, oolong tea, and/or green tea.

Yet another example of an additional ingredient is a probiotic. When present, the liquid composition may comprise from about 10⁶ to 10¹² colony forming unit (cfu) of a probiotic, and alternatively from about 10⁶ to 10¹⁰ cfu of a probiotic. The probiotic component can be lactic acid bacteria. In one embodiment, the probiotic is selected from the group consisting of bacteria of the genera Bacillus, Bacteroides, Bifidobacterium, Enterococcus (e.g., Enterococcus faecium), Lactobacillus, and Leuconostoc, and combinations thereof. In another embodiment of the invention, the probiotic is selected from bacteria of the genera Bifidobacterium, Lactobacillus, and combinations thereof.

Non-limiting examples of lactic acid bacteria suitable for use herein include strains of Streptococcus lactis, Streptococcus cremoris, Streptococcus diacetylactis, Streptococcus thermophilus, Lactobacillus bulgaricus, Lactobacillus acidophilus (e.g., Lactobacillus acidophilus strain), Lactobacillus helveticus, Lactobacillus bifidus, Lactobacillus casei, Lactobacillus lactis, Lactobacillus plantarum, Lactobacillus rhamnosus, Lactobacillus delbruekii, Lactobacillus thermophilus, Lactobacillus fermentii, Lactobacillus salivarius, Lactobacillus reuteri, Bifidobacterium longum, Bifidobacterium infantis, Bifidobacterium bifidum, Bifidobacterium animalis, Bifidobacterium pseudolongum, and Pediococcus cerevisiae, or mixtures thereof, preferably Lactobacillus salivarius, Bifidobacterium infantis, or mixtures thereof.

Prebiotics which are useful include beet pulp, carob bean, psyllium, citrus pectin, rice bran, locust bean, fructooligosaccharide, inulin, oligofructose, galactooligosaccharide, citrus pulp, mannanoligosaccharides, arabinogalactan, lactosucrose, glucomannan, lactulose, polydextrose, apple pomace, tomato pomace, carrot pomace, cassia gum, xanthan gum, gum karaya, gum talha, gum arabic, cellulose, hemicellulose, cellulose ethers, lignin and combinations thereof.

Andrographis is yet another example of an additional ingredient for use herein. As used herein, the andrographis is a plant of the genus Andrographis, having a limited number of species within this genus largely present in Asia. Only a few of the species are medicinal. In one embodiment, the plant is of the species Andrographis paniculata, which may be referenced as Kalmegh in Ayurvedic medicine.

Coffee extract is a polyphenol. The main constituent of coffee extract is coffeic acid. When coffee extract is present nonlimiting sources of coffee extract include coffee, coffee bean, coffee berry, and/or coffee fruits. When coffeic acid is present nonlimiting sources of coffeic acid include coffee bean, coffee fruits, coffee, tea, berries, rosemary extract, and/or grapes extract.

Turmeric extract is a polyphenol. Turmeric extract is a spice which comprises a main active compound that is curcumin. Curcumin is a bioactive polyphenol plant pigment. Nonlimiting source of turmeric extract for use in the present invention is turmeric.

Blueberry extract is a polyphenol. The blueberry extract is rich in anthocyanins which display antioxidant activity.

Grapeseed extract is a polyphenol. The grape seed extract is rich in procyanidins which display antioxidant activity. Nonlimiting source of grapeseed extract for use in the present invention is grape seed.

A “carotenoid” is a class of pigments occurring in the tissues of higher plants, algae, bacteria and fungi. They are usually yellow to deep red. When a carotenoid is present, the carotenoid is selected from the group consisting of betacarotene, lutein, astaxanthin, zeaxanthin, bixin, lycopene, and mixtures thereof.

Amino acids are the “building blocks” of the body. Besides building cells and repairing tissue, they form antibodies to combat invading bacteria & viruses; they are part of the enzyme & hormonal system; they carry oxygen throughout the body and participate in muscle activity. When an amino acid is present, the amino acid is selected from the group consisting of Lysine, Taurine, Histidine, Carnosine, Alanine, Cysteine, and mixtures thereof.

When an antioxidant is present, the antioxidant may be selected from the group consisting of Vitamin E, CoQ10, and mixtures thereof. Major dietary sources of vitamin E are vegetable oils, margarine and shortening, with nuts, seeds, whole grains and wheat germ providing additional sources. “Vitamin E” includes eight different chemical forms: four tocopherols and four tocotrienols. The most biologically active form of vitamin E is alpha-tocopherol.

Nonlimiting examples of preservative include but are not limited to benzoalkonium chloride, EDTA, benzyl alcohol, sorbic acid, potassium sorbate, parabens, benzoic acid, citric acid, sodium benzoate, and mixtures thereof.

### Sweeteners

The liquid composition may comprise a sweetener to provide sweetness and to provide some body and thickness. Natural or artificial sweeteners may be used. Non-limiting examples of artificial sweeteners are selected from the group consisting of sodium saccharin, acesulfame potassium, sucralose, aspartame, monoammonium glycyrrhizinate, neohesperidin dihydrochalcone, thaumatin, neotame, cyclamates, stevia, and mixtures thereof. Generally, such artificial sweeteners are solids when used in sweetening the liquid composition.

When a sweetener is present in the liquid composition, the liquid composition may comprise from about 0.0001% to about 40% sweetener, from about 0.0001% to about 20% sweetener, alternatively from about from about 0.0001% to about 10% sweetener, alternatively from about from about 0.0001% to about 2% sweetener and alternatively from about 0.05% to about 1% sweetener, all by weight of the liquid composition. The sweeteners can be artificial sweeteners.

When an artificial sweetener is present, the liquid composition may comprise from about 0.0001% to about 5% artificial sweetener, from about 0.0001% to about 3.5% artificial sweetener, alternatively from about from about 0.0001% to about 2.0% artificial sweetener, alternatively from about from about 0.0001% to about 1.0% artificial sweetener and alternatively from about 0.05% to about 1.0% artificial sweetener, all by weight of the liquid composition.

### Optional Ingredients

The liquid compositions can comprise a wide range of optional ingredients. Nonlimiting examples of optional ingredients include antimicrobial metal salts, optional mildness enhancers, optional stabilizers, abrasives, biological additives, chemical additives, chelants, denaturants, drug astringents, excipient, emulsifiers, topical analgesics, a film former, fragrance compounds, humectants, opacifying agents, plasticizers, propellants, reducing agents, solvents, foam boosters, stabilizers, hydrotropes, solublizing agents, suspending agents (non-surfactant), a solvent, viscosity increasing agents (aqueous and non-aqueous), sequestrants, buffers, keratolytics, and the like, and combinations thereof. Nonlimiting examples of antimicrobial metal salts include zinc, iron, copper, silver, tin, bismuth, and combinations thereof. Nonlimiting examples of excipients include sorbitol, maltitol, mannitol, and combinations thereof. Unless otherwise specified, the liquid compositions may optionally comprise one or more given optional ingredients at concentrations ranging from about 0.001% to about 99%, alternatively from about 0.01% to about 80%, alternatively from about 0.01% to about 50%, alternatively from about 0.01% to about 10%, all by weight of the liquid composition.

### Active Ingredients

The liquid composition can further comprise a wide range of respiratory active ingredients suitable for treating respiratory symptoms. Nonlimiting examples include analgesics, anticholinergics, antihistamines, anti-inflammatories, antipyretics, antitussives, decongestants, expectorants, mucolytics, antivirals, and combinations thereof.

Example of decongestants include: phenylephrine, naphazoline, 1-desoxyephedrine, ephedrine, propylhexedrine, and pseudoephedrine. Example of anticholinergics include: ipratropium, chlorpheniramine, brompheniramine, diphenhydramine, doxylamine, clemastine, and triprolidine. Common analgesics, anti-inflammatories and antipyretics include: ibuprofen, ketoprofen, diclofenac, naproxen, acetaminophen, and aspirin. Example of antivirals include: amantidine, rimantidine, pleconaril, zanamivir, and oseltamivir. Examples of antitussives include codeine, dextromethorphan, chlophedianol and levodropropizine. Examples of expectorants include guaifenesin. Examples of mucolytics include ambroxol and N-acetylcysteine. Examples of antihistamines include diphenhydramine, doxylamine, triprolidine, clemastine, pheniramine, chlorpheniramine, brompheniramine, dexbrompheniramine, loratadine, cetirizine and fexofenadine, levocytirizine, desloratidine, Amlexanox, Alkylamine Derivatives, Cromolyn Sodium, Acrivastine, Ibudilast, Bamipine, Ketotifen, Nedocromil, Omalizumab, Dimethindene, Oxatomide, Pemirolast, Pyrrobutamine, Pentigetide, Thenaldine, Picumast, Tolpropamine, Ramatroban, Triprolidine, Repirinast, Suplatast Tosylate Aminoalkylethers, Tazanolast, Bromodiphenhydramine, Tranilast, Carbinoxamine, Traxanox, Chlorphenoxamine, Diphenylpyaline, Embramine, p-Methyldiphenhydramine, Moxastine, Orphenadrine, Phenyltoloxamine, Setastine, Ethylenediamine Derivatives, Chloropyramine, Chlorothen, Methapyrilene, Pyrilamine, Talastine, Thenyldiamine, Thonzylamine Hydrochloride, Tripelennamine, piperazines, Chlorcyclizine, Clocinizine, Homochlorcyclizine, Hydroxyzine, Tricyclics, Phenothiazines, Mequitazine, Promethazine, Thiazinamium Methylsulfate, Other Tricyclics, Azatadine, Cyproheptadine, Deptropine, Isothipendyl, Olopatadine, Rupatadine, Antazoline, Astemizole, Azelastine, Bepotastine, Clemizole, Ebastine, Emedastine, Epinastine, Levocabastine, Mebhydroline, Mizolastine, Phenindamine, Terfenadine, Tritoqualine.

The liquid composition may comprise an amount of active ingredient in the range of from 0% to about 15%, alternatively 0.0001% to about 10%, alternatively from about 0.001% to about 7%, and alternatively from about 0.01% to about 5%, all by weight of the liquid composition.

### Method of Making

The liquid compositions may be prepared by any known or otherwise effective techniques suitable for providing a composition that provides a therapeutic benefit. In one embodiment, for purposes of illustration only, the respiratory active ingredients, flavors, non-menthol coolants and other cooling agents are pre-dissolved in glycols and ethanol. Separately, mucoadhesive polymers and/or thickening agents are added to water in such as way so as to avoid clumping and the formation of partially hydrated thickener particles, commonly referred to as “fish eyes.” Once the batch is thickened, water soluble ingredients such as buffers, dyes, sweeteners and preservatives are added and dissolved. The glycol-ethanol premix is then added to the mucoadhesive polymers and/or thickening agents water phase. Bulk liquids, such as high fructose corn syrup, sugar solution, sorbitol and/or glycerin are added at the end and the batch mixed until homogeneous. The composition is then packed into appropriate containers, for example, polyethylene terephthalate bottles.

### Kits

In a further embodiment, the invention can comprise a kit. The kit can comprise: a delivery device and a liquid composition contained in the delivery device; wherein the liquid composition comprising a thickening agent; a mucoadhesive polymer; and a non-menthol coolant and wherein the liquid composition provides an overall cooling sensation. The kit may further comprise at least one additional component. The kit may further comprise at least one optional ingredient. The kit may also comprise an additional liquid composition in a full size, a sample size or both. The kit may further comprise an additional composition that coordinates with the liquid composition that is comprised within the delivery device or attached to the outside of the delivery device. For example, if the liquid composition contained in the delivery device is a liquid composition for extended cooling sensation, the coordinating composition and/or liquid composition may be for congestion. As well, if the liquid composition in the delivery device is a liquid composition for relieving sore throat pain the coordinating liquid composition and or composition may be for runny nose, nasal or chest congestion, sneezing, pressure, headache, aches, fever. As well, if the liquid composition in the delivery device is a liquid composition is to provide a soothing effect for a person experiencing a cold the coordinating liquid composition and/or composition may be a vitamin. The kit could also comprise facial tissue in combination with the liquid composition, a hand sanitizer in combination with a liquid composition or a day time kit or a night time kit that depends on the coordinating liquid composition, additional ingredient and/or optional ingredient that is combined with the liquid composition. The kit may further comprise a coupon, rebate, or advertisement. The kit may further comprise a set of instructions. These instructions may also include illustrations.

### Experimental Studies

An experimental study involves the assessment of liquid compositions 391, 428, 337, and 215 with mucoadhesive polymers and/or thickening agents or without mucoadhesive polymers and/or thickening agents and with or without non-menthol coolants versus a liquid composition 672 with mucoadhesive polymers and thickening agents and non-methanol coolant further versus a liquid composition 723 that has no mucoadhesive polymers, thickening agents and with non-menthol coolants to understand the contribution of each variable on the objective differences in coating, cooling, product thickness, cooling sensation and soothing via the expert descriptive sensory panel. The panel will assess the complete battery of questions typically applied to the descriptive panel.

The panel (n=11, trained in Spectrum™ Descriptive Analysis methodology) followed standard respiratory formulation evaluation protocol (slurp a small amount of product through the lips to evaluate product thickness, then fully swallow the sample and evaluate the remaining ballot-indicated attributes) with the provided liquid compositions in fully blinded and randomized sequential monadic fashion (6 product Latin Square design). 30 ml of the compositions were dispensed by non-panelist laboratory operators in capped, 4 oz. Soufflé cups labeled with only a three digit blinding code under standard fluorescent lighting conditions and ambient temperature (70° F.-72° F.). Key sensory attributes analyzed included: product thickness (immediate timepoint only), oral cooling, throat cooling, cooling sensation, and soothing All attributes were measured immediately after swallowing, and (except for thickness) at the following post-swallowing timepoints: 5, 10, 20, 30, 45, and 60 minutes. A minimum washout time of 2 hours was observed between sample evaluations. All attributes are measured on a 0-60 scale with half-unit intervals.

Specifically, the products tested were:

| | Test Code | | | | | |
|---|---|---|---|---|---|---|
| | 672 | 391 | 428 | 337 | 723 | 215 |
| Ingredient | Wt. % | Wt. % | Wt. % | Wt. % | Wt. % | Wt. % |
| Water | 49.9446 | 50.5119 | 49.9958 | 50.4596 | 50.5666 | 50.0137 |
| High fructose corn syrup | 21.6760 | 21.9221 | 21.6981 | 21.8995 | 21.9459 | 21.7059 |
| (77%) | | | | | | |
| Polyethylene glycol 400 | 14.2887 | 14.4510 | 14.3033 | 14.4361 | 14.4666 | 14.3084 |
| Ethyl alcohol (95%) | 7.6057 | 7.6920 | 7.6134 | 7.6841 | 7.7004 | 7.6162 |
| Propylene glycol | 4.0825 | 4.1288 | 4.0867 | 4.1246 | 4.1333 | 4.0881 |
| Povidone K90 | 1.0206 | 0.0000 | 1.0217 | 0.0000 | 0.0000 | 1.0220 |
| Sodium citrate dihydrate | 0.4048 | 0.4094 | 0.4052 | 0.4090 | 0.4098 | 0.4053 |
| Non-menthol Coolants* | 0.0510 | 0.0520 | 0.0510 | 0.0520 | 0.0100 | 0.0000 |
| Flavor | 0.2705 | 0.2731 | 0.2708 | 0.2728 | 0.2070 | 0.1840 |
| Sodium saccharin | 0.2041 | 0.2064 | 0.2043 | 0.2062 | 0.2067 | 0.2044 |
| Citric acid | 0.1527 | 0.1544 | 0.1528 | 0.1543 | 0.1546 | 0.1529 |
| Xanthan gum | 0.1021 | 0.0000 | 0.0000 | 0.1031 | 0.0000 | 0.1022 |
| Sodium benzoate | 0.1021 | 0.1032 | 0.1022 | 0.1031 | 0.1033 | 0.1022 |
| PEG-40 stearate | 0.0510 | 0.0516 | 0.0511 | 0.0516 | 0.0517 | 0.0511 |
| FD&C red #40 | 0.0435 | 0.0440 | 0.0435 | 0.0439 | 0.0440 | 0.0435 |
| FD&C blue #1 | 0.0001 | 0.0001 | 0.0001 | 0.0001 | 0.0001 | 0.0001 |
| Total | 100.0000 | 100.0000 | 100.0000 | 100.0000 | 100.0000 | 100.0000 |

*non-Menthol Coolant levels: 672, 391, 428, 337 have 0.015% WS-3, 0.035% EverCool ™ 180; 723 has 0.01% WS-3; EverCool ™ 180 is available from Givaudan of Cincinnati, OH as a 5% solution of N-(4-cyanomethylphenyl)-ρ-menthanecarboxamide in flavor ingredient cool white grape.

### Process of Making Formulations

### For 672, 215:

Add povidone slowly to water and mix until dissolved. Add citrate buffers, sodium saccharin, sodium benzoate and dyes to aqueous phase and mix until dissolved. Make glycol premix: Combine glycols, ethyl alcohol, non-menthol coolants and flavor. Add xanthan gum and mix until well dispersed. Add glycol premix to aqueous phase. Add high fructose corn syrup. Add PEG-40 stearate and mix until dissolved and batch is homogeneous.

### For 391, 723:

Add citrate buffers, sodium saccharin, sodium benzoate and dyes to water and mix until dissolved. Make glycol premix: Combine glycols, ethyl alcohol, non-menthol coolants and flavor. Add glycol premix to aqueous phase. Add high fructose corn syrup. Add PEG-40 stearate and mix until dissolved and batch is homogeneous.

### For 428:

Add povidone slowly to water and mix until dissolved. Add citrate buffers, sodium saccharin, sodium benzoate and dyes to aqueous phase and mix until dissolved. Make glycol premix: Combine glycols, ethyl alcohol, non-menthol coolants and flavor. Add glycol premix to aqueous phase. Add high fructose corn syrup. Add PEG-40 stearate and mix until dissolved and batch is homogeneous.

### For 337:

Add citrate buffers, sodium saccharin, sodium benzoate and dyes to water and mix until dissolved. Make glycol premix: Combine glycols, ethyl alcohol, non-menthol coolants and flavor. Add xanthan gum and mix until well dispersed. Add glycol premix to aqueous phase. Add high fructose corn syrup. Add PEG-40 stearate and mix until dissolved and batch is homogeneous.

### Results and Conclusions

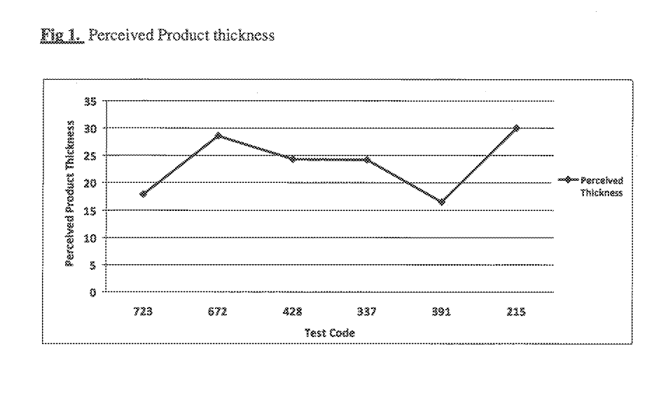

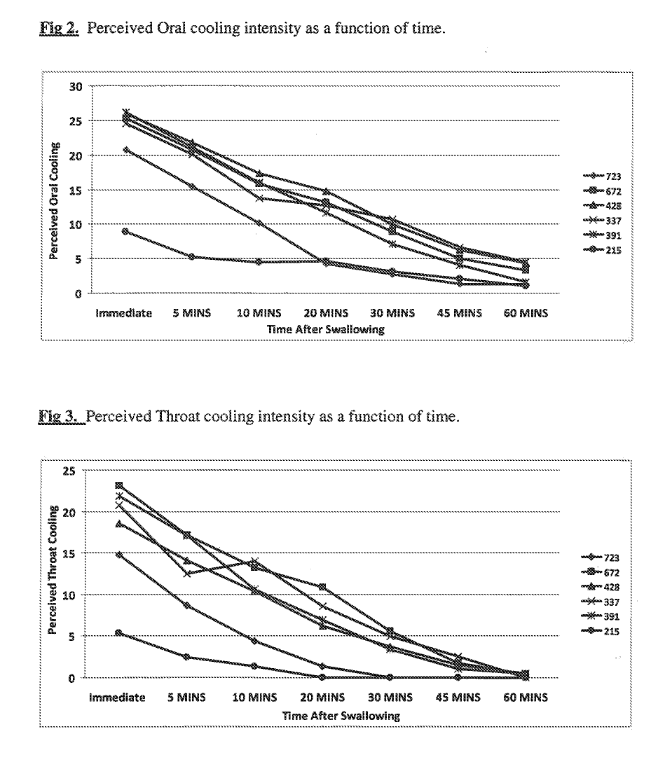

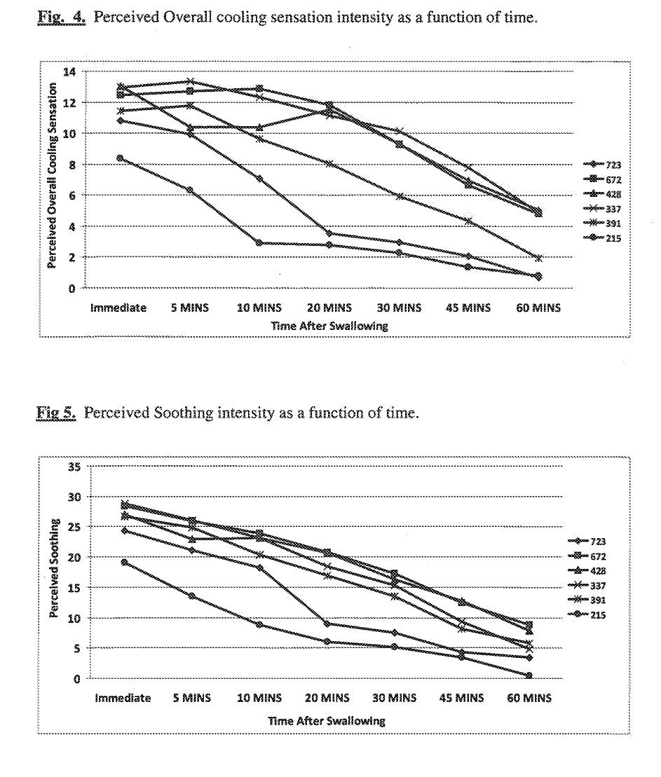

The sensory data, coating, cooling, product thickness, cooling sensation and soothing, collected as part of this Experimental study indicates that the presence of the non-menthol coolant in conjunction with the mucoadhesive polymers and/or thickening agents enhance the sensory perception of several key attributes of the liquid compositions. First, the added mucoadhesive polymers and thickening agents create a greater amount of perceived product thickness of product when the sample is slurped through the lips, See FIG. 1. Both mucoadhesive polymers and thickening agents together created the most perceived product thickness followed by the presence of only one of the either the mucoadhesive polymers and/or thickening agents, followed by the 391 and 723, neither of which contained a polymer. The addition of the non-menthol coolant delivers higher perceived oral cooling to the formulations, with the addition of one or both of the polymers resulting in a higher oral cooling than when the polymers were absent—particularly in the 20-60 minute post-swallowing timeframe, See FIG. 2. For the 10-45 minute post-swallowing timeframe, the addition of thickening agent alone or in conjunction with mucoadhesive polymer caused an increase in perceived throat cooling over the formulations containing only the mucoadhesive polymer or no polymers, See FIG. 3. The perception of cooling sensation is clearly enhanced by the addition of one or both thickening and/or mucoadhesive polymers—especially in the 10-60 minute post-swallowing timeframe, See FIG. 4. Of particular interest is the lack of enhancement of cooling sensation perception in the Immediate to 10 minute post-swallowing timeframe by the mucoadhesive polymer alone, though in later timepoints, the mucoadhesive polymer does provide enhancement to cooling sensation perception over formulations containing no polymers. Finally, the experiential measure of “soothing” (defined by the panel as: “A measure of the perceived feeling of comfort/peace/relief, evaluate as if you had a cold, sore throat, or upper respiratory illness.”, See FIG. 5) is enhanced throughout the evaluation period by the addition of the non-menthol coolants and is higher when one or both of the mucoadhesive and/or thickening agent is present.

Shear Viscosity Method

The liquid compositions have a viscosity which depends upon the applied stress or shear rate. A film falling down a wall subjected to gravity would be a stress-controlled process. In flow curve experiments, the stress applied to a liquid is increased and the resulting viscosity recorded at each stress level. Since the liquid composition will be subjected to body temperature after ingestion, flow curves were measured at 38° C. A TA instruments AR-G2 rheometer equipped with the standard size DIN concentric cylinder (Couette) fixture was used for all testing. The rotor has a diameter of 14 mm and the stator has a diameter of 15 mm. The immersed length of the probe is 42 mm and a gap of 59.2 mm. After the fixture has been installed and the instrument initialized, approximately 13 mL of liquid composition is placed in the Couette cup and the measuring fixture is then lowered into the liquid. After a short temperature equilibration period (2 minutes), the software (TA Version 5.4.0) controls the instrument to perform a stress ramp in a stepped manner from 0.01 Pa to 100 Pa. The viscosity is measured and recorded in a logarithmic spacing of stress over this range. The data is typically plotted as Viscosity vs. Shear Stress. There are several theoretical models that can be fit to the data. The current formulation utilized the Ellis models to represent the liquid compositions tested. The zero-shear viscosity is determined by averaging the viscosity values in the low stress region of the viscosity-stress curve, also called the Newtonian plateau (if present).

Some materials may not display a plateau at low shear rates, making it not possible to determine a zero-shear viscosity. In these cases, it is customary to report the viscosity at a specified rate. Zero shear viscosity is reported in units of centiPoise (cP).

### EXAMPLES

The following examples further describe and demonstrate embodiments within the scope of the present invention. They are given for the purpose of illustration and are not to be construed as limitations of the present invention.

Below are illustrated various non-limiting examples of liquid compositions of the present invention.

| | Example 1 | Example 2 | Example 3 | Example 4 | Example 5 | Example 6 | Example 7 |
|---|---|---|---|---|---|---|---|
| Ingredient | Wt. % | Wt. % | Wt. % | Wt. % | Wt. % | Wt. % | Wt. % |
| Water | QS | QS | QS | QS | QS | QS | QS |
| High fructose | 21.1 | 21 | 21.6 | 22 | 0 | 0 | 18 |
| corn syrup | | | | | | | |
| (77%) | | | | | | | |
| Polyethylene | 14 | 14 | 14 | 14 | 0 | 0 | 17 |
| glycol 400 | | | | | | | |
| Sorbitol (70%) | 0 | 0 | 0 | 0 | 13.1 | 13.1 | 0 |
| Glycerin | 0 | 0 | 0 | 0 | 8 | 8 | 0 |
| Ethyl alcohol | 7.4 | 7.4 | 7.4 | 7.4 | 0 | 0 | 8 |
| (95%) | | | | | | | |
| Propylene | 4 | 4 | 4 | 4 | 23 | 23 | 4 |
| glycol | | | | | | | |
| Acetaminophen | 2 | 2 | 2 | 2 | 2 | 2 | 2 |
| Povidone K90 | 1 | 1 | 0 | 0 | 1 | 0 | 1 |
| Gantrez ® S97 | 0 | 0 | 0.5 | 0 | 0 | 0 | 0 |
| Carbopol ® 971 | 0 | 0 | 0 | 0 | 0 | 0.4 | 0 |
| Sodium citrate | 0.4 | 0.4 | 0.4 | 0.4 | 0.2 | 0 | 0.4 |
| dihydrate | | | | | | | |
| Flavor | 0.2 | 0.2 | 0.2 | 0.2 | 0.3 | 0.3 | 0.1 |
| Menthol | 0.03 | 0.06 | 0.03 | 0.03 | 0.03 | 0.03 | 0.1 |
| WS-3 | 0.02 | 0.02 | 0.02 | 0.02 | 0.02 | 0.02 | 0 |
| White grape | 0.04 | 0.0600 | 0.04 | 0.04 | 0.04 | 0.04 | 0.04 |
| flavor | | | | | | | |
| (EverCool ™ | | | | | | | |
| 180)* | | | | | | | |
| Sodium | 0.2 | 0.2 | 0.2 | 0.2 | 0.2 | 0.2 | 0.2 |
| saccharin | | | | | | | |
| Citric acid | 0.15 | 0.15 | 0.15 | 0.15 | 0.2 | 0.0000 | 0.2 |
| Xanthan gum | 0.1 | 0.1 | 0.1 | 0.1 | 0.1 | 0 | 0.1 |
| Carageenan | 0 | 0 | 0 | 0.1 | 0 | 0 | 0 |
| Sodium | 0.1 | 0.1 | 0.1 | 0.1 | 0.1 | 0.1 | 0.1 |
| benzoate | | | | | | | |
| Sodium | 0 | 0 | 0 | 0 | 0 | 0.1 | 0 |
| hydroxide | | | | | | | |
| (20%) | | | | | | | |
| Dextromethorphan | 0.1 | 0.1 | 0.1 | 0.1 | 0.06 | 0.06 | 0.1 |
| hydrobromide | | | | | | | |
| FD& C Dye | 0.04 | 0.04 | 0.04 | 0.04 | 007 | 007 | 0.04 |
| PEG-40 stearate | 0.05 | 0.05 | 0.05 | 0.05 | 0.05 | 0.05 | 0.05 |
| Doxylamine | 0.04 | 0.04 | 0.04 | 0.04 | 0 | 0 | 0.04 |
| succinate | | | | | | | |
| Phenylephrine | 0 | 0 | 0 | 0 | 0.03 | 0.03 | 0 |
| hydrochloride | | | | | | | |

*EverCool ™ 180 is available from Givaudan of Cincinnati, OH as a 5% solution of N-(4-cyanomethylphenyl)-ρ-menthanecarboxamide in flavor ingredient cool white grape.

Examples 1 and 2 can be made by first slowly adding Povidone to water under good agitation and mix until dissolved. Next, Xanthan gum is then slowly added to the batch under good agitation and mixed until the batch clears and thickens. Buffers, dyes, saccharin and sodium benzoate are added to the batch and mixed until dissolved. Separately, propylene glycol, polyethylene glycol, flavors, cooling agents, non-menthol coolants and ethanol are combined to form a glycol premix. Acetaminophen, dextromethorphan hydrobromide and doxylamine succinate are added to this glycol premix and mixed until dissolved. This glycol premix is then added to the water phase and mixed until homogeneous. High fructose corn syrup is added and mixed until homogeneous. PEG-40 stearate is added and mixed until dissolved.

Example 3 can be made by first slowly adding Gantrez® and xanthan gum to water under good agitation and mixed until the batch clears and thickens. Next, Buffers, dyes, saccharin and sodium benzoate are added to the batch and mixed until dissolved. Separately, propylene glycol, polyethylene glycol, flavors, cooling agents, non-menthol coolants and ethanol are combined to form a glycol premix. Acetaminophen, dextromethorphan hydrobromide and doxylamine succinate are added to this glycol premix and mixed until dissolved. This glycol premix is then added to the water phase and mixed until homogeneous. High fructose corn syrup is added and mixed until homogeneous. PEG-40 stearate is added and mixed until dissolved.

Example 4 can be made by first slowly adding carrageenan and xanthan gum to water under good agitation and mixed until the batch clears and thickens. Buffers, dyes, saccharin and sodium benzoate are added to the batch and mixed until dissolved. Separately, propylene glycol, polyethylene glycol, flavors, cooling agents, non-menthol coolants and ethanol are combined to form a glycol premix. Acetaminophen, dextromethorphan hydrobromide and doxylamine succinate are added to this glycol premix and mixed until dissolved. This glycol premix is then added to the water phase and mixed until homogeneous. High fructose corn syrup is added and mixed until homogeneous. PEG-40 stearate is added and mixed until dissolved.

Example 5 can be made by first slowly adding Povidone to water under good agitation and mixed until dissolved. Xanthan gum is then slowly added to the batch under good agitation and mixed until the batch clears and thickens. Buffers, dye, saccharin, sodium benzoate and phenylephrine are added to the batch and mixed until dissolved. Separately, propylene glycol, cooling agents, non-menthol coolants and ethanol are combined to form a glycol premix. Acetaminophen and dextromethorphan hydrobromide are added to this glycol premix and mixed until dissolved. This glycol premix is then added to the water phase and mixed until homogeneous. Sorbitol and glycerin are added and mixed until homogeneous. PEG-40 stearate is added and mixed until dissolved.

Example 6 can be made by first slowly adding Carbopol® to water under good agitation and mixed until dissolved. Sodium hydroxide (20%) is slowly added to the batch to neutralize/fully thicken the Carbopol®. Sorbitol and glycerin are added to the batch and mixed until homogeneous. Dye, saccharin, sodium benzoate and phenylephrine are added to the batch and mixed until dissolved. Separately, propylene glycol, cooling agents, non-menthol coolants and ethanol are combined to form a glycol premix. Acetaminophen and dextromethorphan hydrobromide are added to this glycol premix and mixed until dissolved. This glycol premix is then added to the water phase and mixed until homogeneous. PEG-40 stearate is added and mixed until dissolved.

Example 7 is made in accordance with standard procedures, optionally in accordance with procedures described hereinabove.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of treating a respiratory symptom in a mammal in need of such treatment comprising the steps of: administering a liquid composition comprising a thickening agent; and a non-menthol coolant; and contacting the liquid composition with the oral mucosa of the mammal wherein the liquid composition is a non-Newtonian liquid that exhibits zero shear viscosity from about 600 to about 1,000,000 cps at a temperature of 37±1° C.

2. The method of claim 1 wherein the thickening agent is selected from the group consisting of xanthan gum, cellulosic polymers, carrageenan, polyacrylic acid, cross-linked polyacrylic acid, polycarbophil, alginate, clay, and combinations thereof.

3. The method of claim 1 wherein the composition comprises from about 0.01% to about 10% of the thickening agent, by weight of the liquid composition.

4. The method of claim 1 wherein the composition comprises from about 0.05% to about 5% of the thickening agent, by weight of the liquid composition.

5. The method of claim 1 wherein the thickening agent is xanthan gum.

6. The method of claim 1 wherein the composition further comprises a mucoadhesive polymer selected from the group consisting of polyvinylpyrrolidone (Povidone), methyl vinyl ether copolymer of maleic anhydride, guar gum, gum tragacanth, polydextrose, cationic polymers, poly(ethylene oxide), poly(ethylene glycol), poly(vinyl alcohol), poly(acrylic acid), cross-linked polyacrylic acid, polycarbophil, poly(hydroxyl ethyl methacrylate), chitosan, cellulosic polymers, and combinations thereof.

7. The method of claim 6 wherein the composition comprises from about 0.01% to about 10% of the mucoadhesive polymer, by weight of the liquid composition.

8. The method of claim 6 wherein the composition comprises from about 0.05% to about 5% of the mucoadhesive polymer, by weight of the liquid composition.

9. The method of claim 6 wherein the mucoadhesive polymer is polyvinylpyrrolidone.

10. The method of claim 6 wherein the non-menthol coolant is selected from the group consisting of menthone glycerol acetal; N-(4-cyanomethylphenyl)-ρ-menthanecarboxamide; N-(2-(pyridin-2-yl)ethyl-3-ρ-menthanecarboxamide; N-(4-sulfamoylphenyl)-ρ-menthanecarboxamide; N-(4-cyanophenyl)-ρ-menthanecarboxamide; N-(4-acetylphenyl)-ρ-menthanecarboxamide, N-(4-hydroxymethylphenyl)-ρ-menthanecarboxamide; N-(3-hydroxy-4-methoxyphenyl)-ρ-menthanecarboxamide; 2-Isopropyl-N,2,3-trimethylbutyramide; N-Ethyl-ρ-menthane-3-carboxamide; Ethyl 3-(ρ-menthane-3-carboxamido)acetate; menthyl lactate; Menthoxypropane-1,2-diol; ρ-Menthane-3,8-diol; Isopulegol, (1R,2S,5R)-2-isopropyl-5-methyl-N-(2-(pyridyn-2-yl)ethylcyclohexane carboxamide, 1-glyceryl-p-mentane-3-carboxylate, ethyleneglycol-p-methane-3-carboxylate, N-t-butyl-p-menthane-3-carboxamide, N-(4-,ethoxyphenyl)-p-menthane-3-carboxamide, 3-(1-menthoxy)propane-1,2-diol, 3-(1-Menthoxy)-2-methylpropane-1,2-diol, menthyl pyrrolidone carboxylate, (1R,3R,4S)-3-menthyl-3,6-dioxaheptanoate, (1R,2S,5R)-3-menthyl methoxyacetate, (1R,2S,5R)-3-menthyl 3,6,9-trioxadecanoate, (1R,2S,5R)-menthyl 11-hydroxy-3,6,9-trioxaundecanoate, (1R,2S,5R)-3-menthyl (2-hydroxyethoxy)acetate, 1-[2-hydroxyphenyl]-4-[2-nitrophenyl-]-1,2,3,6-tetrahydropyrimidine-2-one, 4-methyl-3-(1-pyrrolidinyl)-2[5H]-furanone, menthyl lactate, menthone glycerin acetal, L-Monomenthyl succinate, L-monomenthyl glutarate, 3-1-menthoxypropane-1,2-dio12-1-menthoxyethanoland mixtures thereof.

11. The method of claim 1 wherein the non-menthol coolant is selected from the group consisting of menthone glycerol acetal; N-(4-cyanomethylphenyl)-ρ-menthanecarboxamide; N-(2-(pyridin-2-yl)ethyl-3-p-menthanecarboxamide; N-(4-sulfamoylphenyl)-ρ-menthanecarboxamide; N-(4-cyanophenyl)-ρ-menthanecarboxamide; N-(4-acetylphenyl)-ρ-menthanecarboxamide, N-(4-hydroxymethylphenyl)-ρ-menthanecarboxamide; N-(3-hydroxy-4-methoxyphenyl)-ρ-menthanecarboxamide; 2-Isopropyl-N,2,3-trimethylbutyramide; N-Ethyl-ρ-menthane-3-carboxamide; Ethyl 3-(ρ-menthane-3-carboxamido)acetate; menthyl lactate; Menthoxypropane-1,2-diol; ρ-Menthane-3,8-diol; Isopulegol, (1R,2S,5R)-2-isopropyl-5-methyl-N-(2-(pyridyn-2-yl)ethylcyclohexane carboxamide, 1-glyceryl-p-mentane-3-carboxylate, ethyleneglycol-p-methane-3-carboxylate, N-t-butyl-p-menthane-3-carboxamide, N-(4-,ethoxyphenyl)-p-menthane-3-carboxamide, 3-(1-menthoxy)propane-1,2-diol, 3-(1-Menthoxy)-2-methylpropane-1,2-diol, menthyl pyrrolidone carboxylate, (1R,3R,4S)-3-menthyl-3,6-dioxaheptanoate, (1R,2S,5R)-3-menthyl methoxyacetate, (1R,2S,5R)-3-menthyl 3,6,9-trioxadecanoate, (1R,2S,5R)-menthyl 11-hydroxy-3,6,9-trioxaundecanoate, (1R,2S,5R)-3-menthyl (2-hydroxyethoxy)acetate, 1-[2-hydroxyphenyl]-4-[2-nitrophenyl-]-1,2,3,6-tetrahydropyrimidine-2-one, 4-methyl-3-(1-pyrrolidinyl)-2[5H]-furanone, menthyl lactate, menthone glycerin acetal, L-Monomenthyl succinate, L-monomenthyl glutarate, 3-1-menthoxypropane-1,2-dio12-1-menthoxyethanoland mixtures thereof.

12. The method of claim 1 wherein the liquid composition comprises from about 0.0005% to about 2.0% of the non-menthol coolant, by weight of the liquid composition.

13. The method of claim 1 wherein the liquid composition comprises from about 0.001% to about 1% of the non-menthol coolant, by weight of the liquid composition.

14. The method of claim 1, wherein the non-menthol coolant is N-(4-cyanomethylphenyl)-p-menthanecarboxamide.

15. A liquid composition comprising a thickening agent and N-(4-cyanomethylphenyl)-ρ-menthanecarboxamide.

16. The liquid composition of claim 15 which is a non-Newtonian liquid that exhibits zero shear viscosity from about 100 to about 1,000,000 cps at a temperature of 37±1° C.

17. The liquid composition of claim 15 further comprising at least one additional component selected from the group consisting of tea extracts, Vitamin A, Vitamin C, Vitamin B, Vitamin D, carotenoids, rosemary, rosemary extracts, caffeic acid, coffee extract, tumeric extract, curcumin, blueberry extract, grapeseed extract, rosemaric acid, antioxidants, amino acids, enzymes, prebiotics, probiotics, andrographis extract, 1-tryptophan, Allium sativum, herbal remedies, vitamins, supplements, antioxidants, natural ingredients, minerals, energy boosting ingredients, sleep aids, immune system boosting agents, colorants, preservatives, fragrances, flavorants, fruit extract, salivating stimulators, cooling agents, warming agents, flavoring agents, salivating agents, and combinations thereof.

18. The liquid composition of claim 15 further comprising an active ingredient selected from the group consisting of analgesics, anticholinergics, antihistamines, anti-inflammatories, antipyretics, antitussives, decongestants, expectorants, mucolytics, and combinations thereof.

19. The liquid composition of claim 18 further comprising at least one ingredient selected from the group consisting of excipients, emulsifiers, topical analgesics, film formers, fragrance compounds, humectants, opacifying agents, plasticizers, propellants, reducing agents, solvents, foam boosters, stabilizers, hydrotropes, solublizing agents, suspending agents (non-surfactant), solvents, viscosity increasing agents (aqueous and non-aqueous), sequestrants, buffers, keratolytics, and combinations thereof.

20. A liquid composition comprising a mucoadhesive polymer, a thickening agent and N-(4-cyanomethylphenyl)-ρ-menthanecarboxamide.

21. The liquid composition of claim 20 which is a non-Newtonian liquid that exhibits zero shear viscosity from about 100 to about 1,000,000 cps at a temperature of 37±1° C.
